(19) United States
(12) Patent Application Publication
(10) Pub. No.: US 2022/0027813 A1
(43) Pub. Date: Jan. 27, 2022

(54) RISK IDENTIFICATION AND RESPONSE FOR MITIGATING DISEASE TRANSMISSION

(71) Applicant: Health Solutions Research, Inc., Rockville, MD (US)

(72) Inventors: Ajay Kumar Gupta, Potomac, MD (US); Ramani Peruvemba, McLean, VA (US)

(21) Appl. No.: 17/498,479

(22) Filed: Oct. 11, 2021

Related U.S. Application Data
(63) Continuation-in-part of application No. 17/364,677, filed on Jun. 30, 2021.
Application No. 17/364,677 is a continuation-in-part of application No. 17/107,407, filed on Nov. 30, 2020.
Application No. 17/107,407 is a continuation-in-part of application No. 16/887,608, filed on May 29, 2020.
Application No. 16/887,608 is a continuation-in-part of application No. 16/429,550, filed on Jun. 3, 2019.
Application No. 16/429,550 is a continuation-in-part of application No. 16/126,537, filed on Sep. 10, 2018.
Application No. 16/126,537 is a continuation-in-part of application No. 16/024,387, filed on Jun. 29, 2018, now Pat. No. 11,177,040.
(60) Provisional application No. 62/602,661, filed on May 1, 2017.

(51) Int. Cl. G06Q 10/06; G16H 50/80; G16H 50/20

(52) U.S. Cl. CPC G06Q 10/0635; G16H 50/20; G16H 50/80

(57) ABSTRACT

Determining and tracking spread of at least one disease at a plurality of points of interests that includes generating a geocoding movement data and health information of a plurality of subjects acquired from a plurality of sources, merging the geocoded movement data and the geocoded health information, and obtaining point of interest information of the plurality of points of interest from the plurality of sources. Predicting transmission risk at a point of interest further includes developing, for each of the plurality of points of interest, a business risk index of the risk of transmission of the at least one disease at the plurality of points of interest based on the merged geocoded movement data, the geocoded health information, and the point of interest information, outputting the business risk index for the plurality of points of interest, and developing and adjusting strategies to mitigate the disease based on the business risk index.

### CROSS-REFERENCE TO RELATED APPLICATIONS

This application is a continuation-in-part of U.S. application Ser. No. 17/364,677, filed Jun. 30, 2021; which is a continuation-in-part of U.S. application Ser. No. 17/107,407, filed Nov. 30, 2020; which is a continuation-in-part of U.S. application Ser. No. 16/887,608, filed May 29, 2020; which is a continuation-in-part of U.S. application Ser. No. 16/429,550, filed on Jun. 3, 2019; which is a continuation-in-part of U.S. application Ser. No. 16/126,537, filed on Sep. 10, 2018; which is a continuation-in-part of U.S. application Ser. No. 16/024,387, filed on Jun. 29, 2018, which claims the benefit of provisional application 62/602,661, filed May 1, 2017, the entire disclosures of which are hereby incorporated by reference.

### FIELD OF THE DISCLOSURE

The present disclosure is generally related to identifying the risk associated with disease transmission at points of interest and response thereof; and in one or more example embodiments, to identifying what point of interest or groups of points of interests are at greater risk for disease transmission and responding accordingly.

### BACKGROUND

Infectious and non-infectious diseases may occur on a scale that crosses regional, state, country, and/or international boundaries, affecting a substantial number of people. For example, there have been a number of pandemics, including the COVID-19 pandemic of 2019, that affected millions of people world-wide. Strategies for controlling the scope of such a pandemic include containment, mitigation, and suppression. But, such strategies have led to unintended negative consequences, for example, social isolation, damage to the economy, etc.

Containment is typically undertaken in the early stages of a pandemic, and may include contact tracing and isolating infected individuals to stop the disease from spreading to the rest of the population. When it is not possible to contain the spread of the disease, strategic mitigation strategies may be implemented, by which measures may be taken to slow the spread of the disease and mitigate its effects on society, including burdens on the healthcare system. Suppression requires more extreme long-term interventions so as to reverse the pandemic by reducing the basic reproduction number. The suppression strategy, which includes stringent population-wide social distancing, business closures, home isolation of cases, and household quarantine, carries with it considerable social and economic costs.

### SUMMARY

The embodiments described, recited, and suggested herein mitigate, determine and track, and/or control the transmission of a disease in a manner that does not require closing or restricting all or certain segments of a community, since blanket closures of particular points of interests, such as, businesses, restaurants, hotels, grocery stores, malls, parks, buildings, attractions, houses of worship, etc., are not good for the social health of the community and/or the economy, e.g., due to isolation and economic costs of shutting down businesses.

In accordance with at least one example embodiment, a method to determine and track spread of at least one disease at a plurality of points of interest includes: geocoding movement data and health information of a plurality of subjects acquired from a plurality of sources; merging the geocoded movement data and the geocoded health information; obtaining point of interest information of the plurality of points of interest from the plurality of sources; developing, for each of the plurality of points of interest, a business risk index of the risk of transmission of the at least one disease at the plurality of points of interest based on the merged geocoded movement data, the geocoded health information, and the point of interest information; outputting the business risk index for the plurality of points of interest; and developing and adjusting strategies to mitigate the disease based on the business risk index.

In accordance with a second non-limiting embodiment, a system for determining and tracking spread of at least one disease at a plurality of points of interest includes: a memory to store structured information and unstructured information; and a processor configured to: geocode movement data of a plurality of subjects acquired from a plurality of sources; geocode health information of the plurality of subjects from the plurality of sources; merge the geocoded movement data and the geocoded health information; obtain point of interest information of the plurality of points of interest from the plurality of sources, develop, for each of the plurality of points of interest, a business risk index of the risk of transmission of the at least one disease at the plurality of points of interest based on the merged geocoded movement data, the geocoded health information, and the point of interest information; output the business risk index for the plurality of points of interest; and request a user to develop and adjust strategies to mitigate the disease based on the business risk index.

In accordance with a third non-limiting embodiment, a non-transitory computer-readable medium having computer-readable instructions that, when executed by a computing device, causes the computing device to perform operations including geocoding movement data of a plurality of subjects acquired from a plurality of sources; geocoding health information of the plurality of subjects from the plurality of sources; merging the geocoded movement data and the geocoded health information; obtaining point of interest information of the plurality of points of interest from the plurality of sources, developing, for each of the plurality of points of interest, a business risk index of the risk of transmission of the at least one disease at the plurality of points of interest based on the merged geocoded movement data, the geocoded health information, and the point of interest information; outputting the business risk index for the plurality of points of interest; and developing and adjusting strategies to mitigate the disease based on the business risk index.

It will be appreciated that the above embodiments are merely illustrative of the technical concepts and features of the response engine for determining the business risk, and these embodiments are to provide a person skilled in the art with an understanding of the contents of the response engine in order to implement the response engine without limiting the scope of protection of the response engine. Any features described in one embodiment may be combined with or incorporated/used into the other embodiment, and vice versa. The equivalent change or modification according to the substance of the response engine should be covered by the scope of protection of the response engine.

It will be appreciated that the response engine disclosed herein may provide a predictive tracking and modelling tool for determining and tracking the spread of a disease, e.g., infectious or non-infectious, at particular points of interest. By leveraging the geospatial tracking capabilities, artificial intelligence based predictive analytics, and health, social, and environmental data sets, the response engine may track the disease dynamics and identify at-risk points of interest that have contributed, or may contribute, to the potential spread of the disease. The modelling may follow a phased approach, e.g., local, regional, national, international, and global, that expands both the geographic region and improves accuracy of the prediction in each successive phase to allow granular assessment at the national, regional, and local levels. The response engine may allow response managers and/or decisions makers to implement mitigation strategies ahead of the movement of disease, as opposed to reactive measures that at times prove to be insufficient in halting the spread of the disease. It will also be appreciated that the output of the response engine, e.g., business risk indices, etc., may be integrated into software systems supporting disaster and emergency response management platforms. The business risk indices may include the average dwelling time, number of visitors, etc., at a point of interest and may be integrated into, for example, a data processing system such as an emergency response management system, manager of a point of interest, supply chain management system, health information system platform or dashboard, or any other suitable data processing system.

It will further be appreciated that the response engine described, recited, and even suggested herein may assist and/or facilitate disease mitigation efforts. The response engine may identify points of interests that have contributed to disease transmission, e.g., reverse contract tracing, and/or are likely to contribute to the transmission of the disease, as well as assess environmental factors that may potentially exacerbate or inhibit the spread of the disease, such as the incubation temperature, humidity, and other environmental factors that exacerbate viral replication. The response engine may produce recommendations or automatically trigger mitigation responses/strategies for particular points of interest in a community. For example, it is appreciated that while contract tracing is available to track the spread of a disease on a small scale, e.g., tracing where people have visited points of interests for a limited group, once the disease has spread to the community level, it is difficult to contract trace effectively and maintain the privacy of the person reporting the contact tracing. The business risk index, however, may be used for reverse contact tracing by viewing historical data to see where the disease was likely spread. For example, the historical data may include a modified Wells-Riley model of a disease to determine, for example, viral load, and transmission characteristics and use the mobility data to determine how many people were likely infected at particular points of interests. By determining how many people were likely infected, the privacy implications are removed, since individual people do not have to provide such data to local, state, and national governments.

By accurately assessing what points of interests are/were least conducive to the growth and spread of the disease, prudent decisions may be made as to when mobility levels and the associated economic activity may return to normal levels, e.g., determinations as to when and where resumption of social interaction constitutes an acceptable risk. Accordingly, the response engine may facilitate upward and downward scaling of the mitigation/de-mitigation efforts as the number of people under quarantine expands and eventually contracts.

The response engine may further facilitate generating mitigation and de-mitigation responses/strategies plans in a structured and orderly manner. That is, the response engine may facilitate the management of large-scale social isolation by balancing the risk of contagion against the risk of adverse mental health consequences for individuals and economic loss by communities at-large. The response engine may be leveraged in future pandemic situations and in other cities and regions to guide quarantine response.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries, e.g. boxes, groups of boxes, or other shapes, in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

The present disclosure provides a detailed and specific description that refers to the accompanying drawings. The drawings and specific descriptions of the drawings, as well as any specific or alternative embodiments discussed, are intended to be read in conjunction with the entirety of this disclosure. The response engine may, however, be embodied in many different forms and should not be construed as being limited to the embodiments set forth herein; rather, these embodiments are provided by way of illustration only and so that this disclosure will be thorough, complete and fully convey understanding to those skilled in the art.

FIG. 1 is a schematic diagram of an advanced smart response engine, according to at least one example embodiment described herein.

FIG. 2 is a schematic diagram of a processing system, according to at least one example embodiment described herein.

FIGS. 3A and 3B show an example map and output generated by an advanced smart response engine, according to at least one example embodiment described herein.

FIG. 4 shows an example processing flow for a response engine to generate a business risk model, according to at least one example embodiment described herein.

FIG. 5 shows an example processing flow for a response engine to facilitate generating mitigation strategies and/or de-mitigation strategies, according to at least one example embodiment described herein.

FIG. 6 illustrates at least one computer program product that may be utilized to provide an advanced smart response engine, according to at least one example embodiment described herein.

FIG. 7 shows a block diagram illustrating an example computing device by which various example solutions described herein may be implemented, according to at least one example embodiment described herein.

FIG. 8 illustrates a work flow of an advanced smart response engine, according to at least one example embodiment described herein.

References are made to the accompanying drawings that form a part of this disclosure and which illustrate embodiments in which the systems and methods described in this specification may be practiced.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a”, “an” and “the” include plural references unless the context clearly dictates otherwise. Although any systems and methods similar or equivalent to those described herein may be used in the practice or testing of embodiments of the present disclosure, the preferred, systems and methods are now described.

It will be appreciated that “risk area” or “risk zone” may refer to point(s) of interest(s) where people may be at risk of being affected by a particular disease to varying degrees. The terms “high risk area” and/or “hot zone” may refer to a risk area or a risk zone where the number of people that may be at risk of being affected by the disease relative to the overall population of the area is greater than a predetermined value (e.g., 5% within a city/town, a county, a State, etc.). The terms “low risk area” and/or “low risk zone” may refer to a risk area or a risk zone where the number of people that may be at risk of being affected by the disease relative to the population of the area is equal to or lower than a predetermined value (e.g., 5% within a city/town, a county, a State, etc.). The scores/indices may be quantified (e.g., 1-5, 1-100, etc., with the higher the scores/indices indicating areas of higher risk). For example, when the risk score is 3 or more over 5 (if the maximum score is 5) (or 60 or more over 100 (if the maximum score is 100)) for an area, the area is a high risk area (hot zone); when the risk score is less than 3 over 5 (or less than 60 over 100) for an area, the area is a low risk area/zone. It is appreciated that the quantification of the risk may include multiple ranges of categories, e.g., high, medium, low, or more levels, such as seven different levels.

Embodiments of the present disclosure will be described more fully hereafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

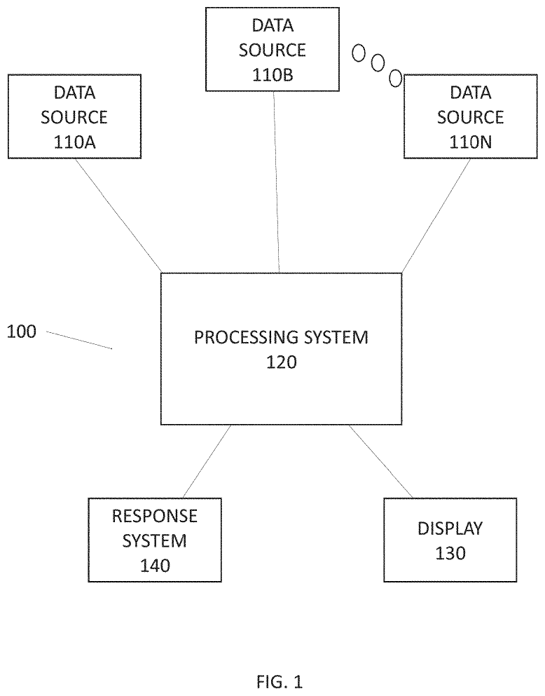

FIG. 1 is a schematic diagram of an embodiment of a response engine 100 that identifies business risks of a plurality of points of interests, e.g., businesses, such as restaurants, hotels, grocery stores, malls, parks, buildings, attractions, houses of worship, or other places where people congregate. The response engine 100 may be used for determining and tracking infectious and non-infectious diseases and/or managing and responding to the disease, such as determining and tracking a spread of the disease and evaluating response resources, e.g., predicting risk areas of the disease and how best to mitigate transmission of the disease. FIG. 1 shows a plurality of health and non-health data sources 110A, 110B, . . . 110N, which may be communicatively coupled to a processing system 120. Processing system 120 may be communicatively coupled to a display 130 and a response system 140, e.g., a response manager or decision maker (e.g., local or state government agency, manager of a point of interest, public health officials, etc.). By way of example, one or more of the communicative couplings may be wired or wireless communications.

Data sources 110A, 110B, . . . 110N may refer to, but not be limited to databases having disease information, population mobility data, environmental, and point of interest data. The disease databases may include local, state, and national health departments and health resources, CDC Wonder, World Health Organization (WHO), The United Nations, Kaiser Family Foundation, other non-profits, and transmission risk indices and/or vectors of transmission for particular diseases, e.g., how a disease is spread. The population mobility data may include mobility and foot traffic data for a given locality, state, region, or country based on, for example, cell phone data, sales data, satellite data, Wi-Fi signals, automobile data, proximity/pressure sensors, cameras, manual counters, and many other potential sources either individually or in combination. The point of interest data may include point of interest layout and geospatial visualizations, physical dimensions and/or geodesic area, category type of the point of interest, e.g., restaurant, retail, parks, museums, bank, hotels, grocery stores, malls, houses of worship, etc. Further, not only are the systems described, recited, and foreseen herein not limited to the data sources listed above, but they are not limited in quantity to those shown in FIG. 1.

The response engine 100 may analyze the disease information, mobility data, and environmental data to identify health risks in particular areas, e.g., hospital data of infected people or subjects, seasonal changes that may affect if people are social distancing, etc., health risks for certain Census block groups, as well as determine potential solutions for managing control of a disease on various scales. Data sets are geocoded, merged, and analyzed by the response engine 100 to provide pertinent information, such as the identification of future disease hot zone locations; how the disease initially spread; low risk zones that may be re-opened safely, i.e., public gatherings and commercial activity may be resumed, relative to a broad re-opening of the economy; as well as other socioeconomic outcomes. The response engine 100 may identify, e.g., where people may be at risk for contracting the particular disease to varying degrees at varying points of interest, areas at the point of interest that people may be at higher risk for contracting the particular disease, etc.

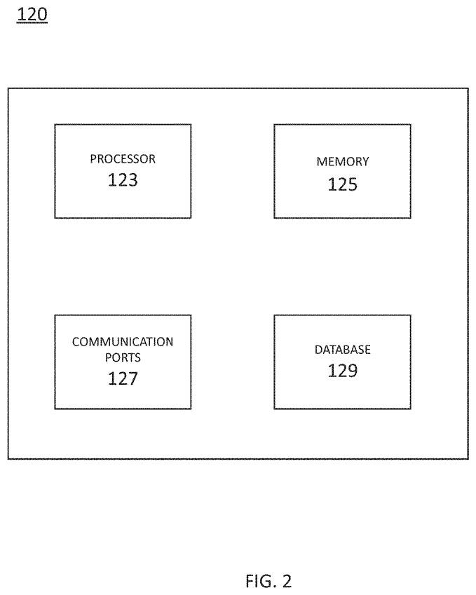

FIG. 2 is a schematic diagram of a processing system 120, according to at least one example embodiment described herein. In one or more embodiments, processing system 120 may include one or more processors or computing devices 123 (collectively, “processor” as used herein), a system memory 125, communication ports 127 to acquire data from one or more of data sources 110A . . . N, and a database 129. Processing system 120 may be configured and arranged to implement an information system platform with a data analytic engine (such as the response engine) as discussed below. Data acquired from the data sources 110A . . . N may be added to the database 129. The stored data may be analysed, merged, and/or geocoded using data analytics, and formatted for output for any suitable purpose, including for display on a geographic or other map via display 130, or for further analysis or review (e.g., personal or machine) either locally or remotely (e.g., into the computer(s) for the response manager or decision maker or web-accessible interactive interface).

In one or more embodiments, based on the data obtained from data sources 110A . . . N, the response engine 100 may determine disease dynamics using a machine learning approach based on the number of confirmed/positive diagnoses (or diagnoses related to whatever health risk is being tracked) on the local or regional level, for example, of a Census block group, the point of interest data, and the mobility data to quantify the risks pertaining to the disease (or health risk) to certain Census block groups or the community at large. The machine learning approach may use a number of different analytic approaches, such as, regression, independent components analysis, such as, K nearest neighbors and last squares, or principle components analysis, or combinations thereof. The response engine 100 provides spatial data infrastructure including geospatial visualization capabilities, machine learning and Artificial Intelligence (AI) based predictive analytics, and data sets from data sources 110A . . . N to determine, for example, average dwelling time, average number of visits, etc.

The response engine 100 may also use transmission risk indices at the state/national/international and local levels to track the disease flow and identify at-risk points of interests that may be susceptible for potential future spread of a disease. The transmission risk indices identifies the high risk points of interests as well as assesses the environmental factors that may potentially exacerbate or inhibit the viral transmission (e.g., incubation temperature for viral replication, airflow, transmission distance, ambient temperature, etc.). The transmission risk indices may be based on the past information of the disease, for example, the points of interest where the disease previously spread, a susceptible-exposed-infectious-removed (SEIR) model, areas at the point of interest that likely contributed to the spread of the disease, types of points of interest where the disease spread, R₀ and/or vector of transmission or disease dynamics, etc. For example, the transmission risk index may be the Transmission Risk Index as disclosed in U.S. publication 2020/0294680 and U.S. publication 2021/0166819, which are incorporated by reference.

The response engine 100 may further generate a Business Risk Index (BRI) based on the transmission risk indices and data sources 110A . . . N to provide the response manager or decision maker, e.g., state or local government or point of interest manager(s), health information related to the potential impact of the disease. That is, the potential impact of the disease includes the risk of infectious and non-infectious disease transmission, and mitigation strategies for a point of interest or a categorized group of points of interest, e.g., restaurants or mall. In an embodiment, the response engine 100 may use the BRI to determine whether a specific point of interest would be impacted, a certain category of points of interests, or a certain Census block group, e.g., smallest geographical unit in which data is collected form a fraction of all households, would be impacted based on the provided health information so that the response manager or decision maker can determine the appropriate response (or non-response) efforts. For example, in an embodiment, if the disease was previously spread at a retail store selling merchandise to children at a local mall, the response engine 100 may determine that the retail store selling merchandise to children would need to shut down, while allowing the remaining retailers and/or restaurants at the local mall to stay open. That is, the response engine 100 not only determines whether a specific point of interest is a risk for spreading a disease, but uses the BRI to determine the likelihood of the spread of the disease based on how the disease previously spread in the community.

The response engine 100 may also determine based on the transmission risk indices, disease dynamics, and/or historical data, the likely contributing factors that contribute to the spread of the disease. For example, the factors that may contribute to the spread of the disease include dwelling time at a point of interest, e.g., how long people or subjects are at a particular point of interest or area in a portion of interest, specific areas at a point of interest where people were found to congregate, e.g., sampling stations, layout of the point of interest, e.g., one ingress/egress for people to enter and leave the point of interest, and other determinates that may be found using statistical analysis that may influence the level of exposure.

For example, in an embodiment, the business risk index may be determined by first determining the transmission risk index of a point of interest at an hour t according to the following:

POI_TRI=sum(TRI_i*number of visitors from location_i)/100.

Then the number of asymptomatic/infected individuals at the point of interest at hour t according to the following:

I=(Cumulative_active/Total population)*visit from a county per hour.

The infection rate at the point of interest may at hour t may then be determined as follows:

Inf_rate=TRI_POI_hour*((medium_dwell_time_hour)\hat{ }2*(I/area_sq_ft)*visit_from_a county_per_hour.

The transmission risk index may then be used to determine the number of potentially susceptible people who visit the point of interest for a particular time frame, e.g., in a day, in a week, in a month, as expressed by the following:

POI_Susceptibles_month=Visitors−(sick+vaccinated+recovered)=Target

POI_Susceptibles_month=(visit_cnty_month−poi_inf_month)*((EP_Cum_Vacc/100)+(PCT_Cum_Recovered/100)) or

POI_Susceptibles_month=visit_cnty_month{visit_cnty_month*[(poi_inf_month/visit_cnty_month)+(EP_Cum_Vacc/100)+(PCT_Cum_Recovered/100)]}

Thus, the potential number of transmissions in the month at the POI of interest may be determined and expressed as the following:

POI_Potential_Transmissions_month=TRANSMISSION_RATE*POI_inf_rate_month*POI_Susceptibles_month

The response engine 100 may use the BRI to determine and/or make recommendations to the decision makers on mitigation strategies or responses that should be used at the point of interest. For example, the mitigation strategies or response may include social distancing or quarantine measures as well as disinfection of certain areas at a point of interest, e.g., points of interest that are hot spots for potential spread, change areas where people dwell or congregate, e.g., eating dessert or having coffee at a different location at the point of interest from where they dined, how long people should dwell or congregate, e.g., move people every 15 minutes, changes and improvements to the built environment, such as update/upgrade ventilation, air exchange, or airflow systems, change hours of operation and/or limit when certain age categories can enter the point of interest, etc.

The response engine 100 may also use the BRI to predict the location of future outbreaks of the disease, and may identify the points of interest(s) or category of points of interest(s) with the highest risk of disease transmission, e.g., has factors that contribute to disease transmission. The prediction by the response engine 100 may be based on current cases, disease progression, disease dynamics, mobility data, and/or which points of interest(s) and/or category is the most visited. The output of the response engine may enable mitigation strategies e.g., social distancing, improved air flow, congregation areas for people, etc. in advance of the outbreaks, rather than chasing the outbreaks, to improve the ability to halt the spread of the contagion without unnecessarily closing all points of interests. That is, the output is used to make targeted intervention without adversely closing down the community or specific group of points of interest, e.g., mall.

The response engine 100 may also use the BRI to automatically trigger a health risk review of a specific point of interest or make changes to operation of the point of interest if or when the BRI exceeds a predetermined threshold, e.g., a ranking of 80 out of 100 of the risk of disease transmission. If the BRI exceeds the predetermined threshold, the response engine 100 may transmit an alert that is displayed to the response manager or decision maker, e.g., display or automatically directs or connects the response manager or decision maker to a link to the point of interest having the high business risk index, and/or automatically instructs the point of interest to change the hours of operation, limit capacity, start social distancing measures, etc. It is appreciated that by automatically controlling the point of interest, the chain of the spread of the disease may be broken based on a designed analytics which is outside the control of influences of politics and the mind-sets of people. It is appreciated that the BRI predetermined threshold may also be used to trigger incremental mitigation strategies. For example, if the BRI of a point of interest reaches 60, then the response engine 100 may implement a social distancing requirement and/or maximum occupant density restriction. If the BRI reaches 80, additional mitigation strategies may be implemented, for example, reducing hours of operation of the point of interest. If the BRI reaches 95, the response engine 100 may instruct the closure of the point of interest. It is also appreciated that while the business risk index is provided as values, the business risk index and responses may be based on relative values based on other points of interests, e.g., the business risk index as compared to other points of interests. For example, if a grocery store has a business risk index of 80, while a clothing retailer has a business risk index of 70, mitigation strategies and responses may be implemented at the grocery store but not necessarily at the clothing retailer to the same level.

These mitigation strategies may be guides, e.g., eventual de-quarantine efforts, to resume economic activity at “safe” or “low risk” points of interests, to reduce the risk of a second bump of cases as normal activity and social interaction is resumed, to speed the safe resumption of normal economic activity and benefit the economy, and to resume normal activities and reduce the mental health risk associated with long-term social isolation. Such mitigation strategies enable decision makers with the ability to provide targeted mitigation strategies to specific hotspots and allow for a controlled reopening or remained opening of the economy, e.g., mitigate personal and professional disruptions caused by blanketed closures.

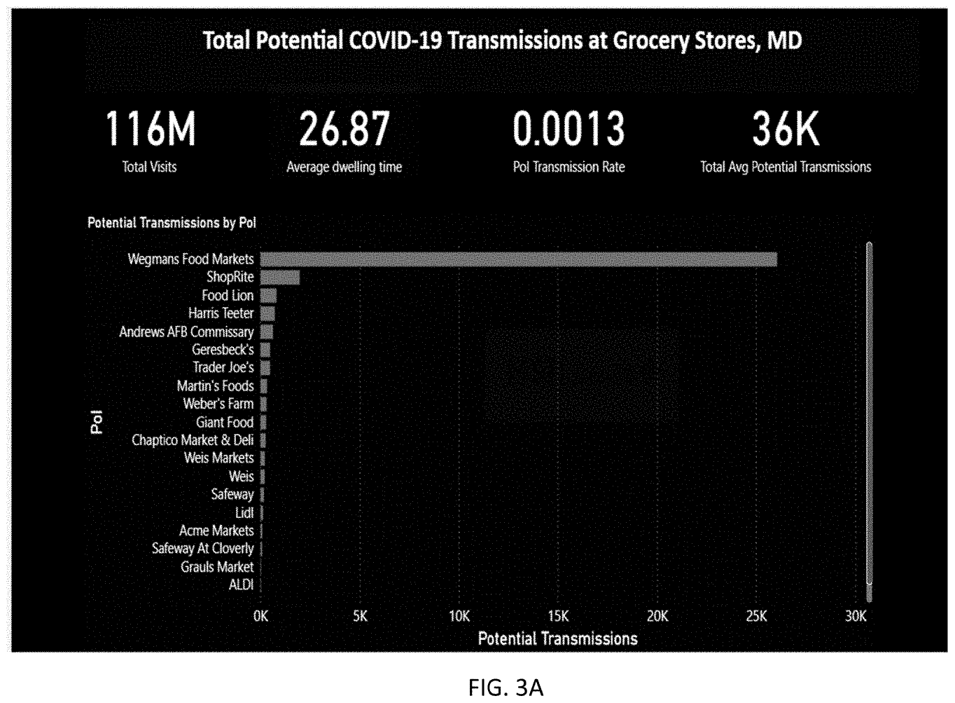

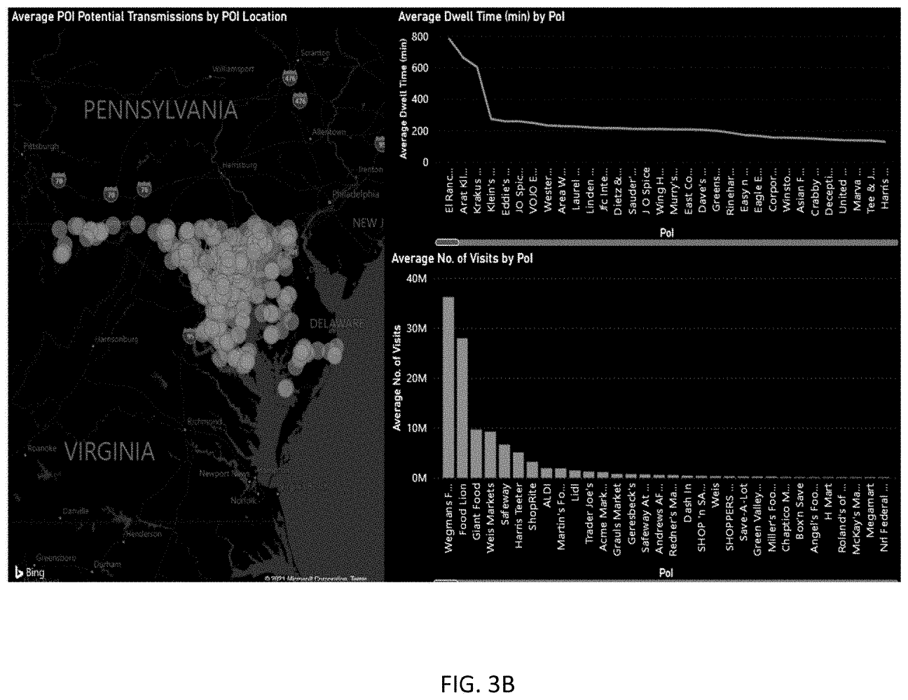

FIGS. 3A and 3B show example graphs and maps generated by a response engine, according to at least one example embodiment described herein ranked in order by the points of interests having the highest business risk index. It is appreciated that other graphs and maps may be produced to visualize the BRI and results of the response engine, as appropriate.

The example map generated by the response engine disclosed herein may show a determination and tracking of a spread of a disease (e.g., SARS, COVID-19) and serve as a tool for assessing mitigation strategies and for predicting points of interest that have the highest risk for transmission of the disease, of varying degrees of risk, and for illustrating hot zones in accordance with at least some embodiments described herein.

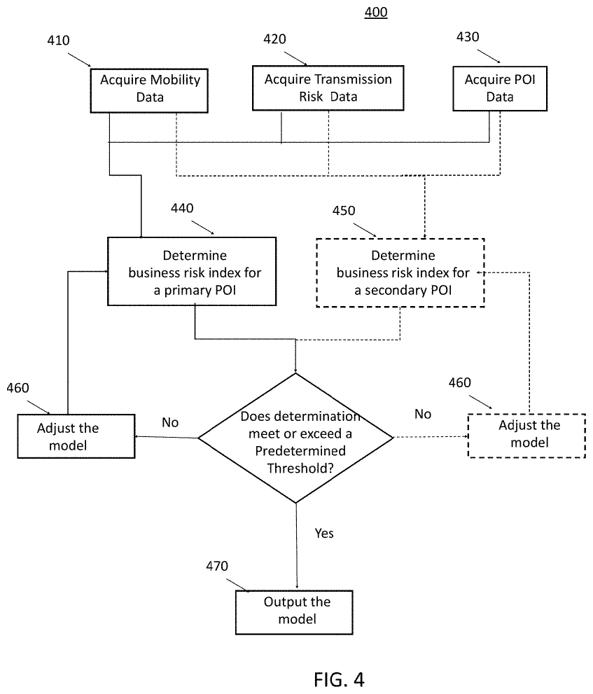

FIG. 4 shows an example processing flow 400 for a response engine to generate a disease predictive model and business risk index for a point of interest, according to at least one example embodiment described herein. In one or more embodiments, the model uses past, current, and/or predictive disease data, mobility data, and point of interest data to determine transmission risk of a disease at the plurality of points of interest.

Processing flow 400 may include one or more operations, actions, or functions depicted by one or more blocks 410, 420, 430, 440, 450, 460, 470, and 480. Although illustrated as discrete blocks, various blocks may be divided into additional blocks, combined into fewer blocks, or eliminated, depending on the desired implementation. As a non-limiting example, the description of processing flow 400, corresponding to the depiction thereof in FIG. 4 and performed by processing system 120 in one or more embodiments described herein, pertains to predicting people or subjects affected by the disease under a certain condition. Processing may begin at blocks 410, 420 and/or 430.

Block 410 (Acquire Mobility Data) may refer to processing system 120 receiving a set of mobility data from data sources 110A . . . 110N via communication ports 127. Mobility data may include mobility and foot traffic data for a given locality, state, region, or country based on, for example, cell phone data, sales data, satellite data, Wi-Fi signals, automobile data, proximity/pressure sensors, cameras, manual counters, etc., including but not limited to wireless or wired communications from data sources 110A . . . N or manual entry (for example, by an operator using a keyboard or tablet, smartphone, etc. utilizing appropriate application software). The mobility data may also include statistics related to Census block groups, total monthly visits, average dwelling time for specific points of interest(s), e.g., individual point of interest or category of point of interests, times when the point of interest(s) are visited, etc. Block 410 may be followed by either of Block 420 and Block 430.

Block 420 (Acquire Transmission Risk Data) may refer to processing system 120 receiving a set of disease data. The disease data may include transmission risk data (e.g., at a geographic area such as a city/town, a state/province, a country, etc.) to identify transmission risk associated with diseases and/or vectors of transmissions, e.g., a susceptible-exposed-infectious-removed (SEIR) model or disease dynamics from the CDC, WHO, etc. from data sources 110A . . . 110N via communication ports 127. Transmission risk data may include a transmission risk index for the infectious disease, e.g., R₀ value, conditions and places that the previous disease spread, types and/or conditions of points of interest that exacerbated the spread of the disease, the same or similar disease, e.g., similar R₀ and/or vector of transmission or disease dynamics, etc., including but not limited to wireless or wired communications from data sources 110A . . . 110N or manual entry (for example, by an operator using a keyboard or tablet, smartphone, etc. utilizing appropriate application software). In some embodiments, and without limitation, the transmission risk data may be obtained from public sources such as the government agency, the Centers for Disease Control, or determined by a response engine etc. Block 420 may be followed by Block 430.

Block 430 (Acquire Point of Interest Data) may refer to processing system 120 receiving a set of point of interest(s) data from data sources 110A . . . 110N via communication ports 127. Point of interest data may include a layout and geospatial visualizations, dimensions and/or geodesic area, mechanical equipment installed at the point of interest, e.g., HVAC system and capacity, air flow patterns, air exchange rate, occupancy/capacity, category type of the point of interest, e.g., restaurant, retail, parks, museums, bank, hotels, grocery stores, malls, houses of worship, etc., including but not limited to wireless or wired communications from data sources 110A . . . 110N or manual entry (for example, by an operator using a keyboard or tablet, smartphone, etc. utilizing appropriate application software). In some embodiments, and without limitation, the points of interest data may be obtained from public sources such as the local or state agencies or online sources. Block 430 may be followed by Block 440.

In one or more embodiments, the data acquired from Blocks 410, 420, and/or 430 may be geocoded. Geocoding may include merging data obtained from disparate sources (structured, unstructured, private, public, demographic, social, socioeconomic, environmental, etc. data) together with their associated geographical locations as a connector between and among them. The criteria for associating particular data with a particular geographical location and/or point of interest may be user-defined or predefined and implemented by processing system 120 in accordance with geographic information included with the acquired data. For example, and without limitation, a user may define a region of interest and manually associate specific acquired data with the region of interest (e.g., by “plotting” the data on a map). As another nonlimiting example, Census data for a predefined region of interest (e.g., the population density of a city, such as Baltimore, Md.) may be automatically plotted on a map. Other association options will be apparent to one of ordinary skill and are properly considered within the scope of one or more of the described embodiments.

Block 440 (Identify Primary Business Risk Index) may refer to processor 123 determining a business risk index for a primary point of interest which is a business risk for the transmission of the disease at the primary point of interest. The business risk index is determined using a network based approach based on at least merging the mobility data and transmission risk data, and the point of interest data to build a model of the business risk for the transmission of the disease. The model may use disease dynamics using a regression based machine learning and Artificial Intelligence (AI) based predictive analytics approach based on the number of confirmed/positive diagnoses (or disease dynamics related to whatever health risk is being tracked) on the local or regional level and the geocoded mobility data and the point of interest data to quantify the risks pertaining to the disease (or health risk). In an embodiment, the model may be a correlation or calculation that results in a value that may be compared to a threshold value, e.g., different weighted factors of the mobility data, transmission risk data, and the point of interest to determine a business risk index value. For example, in one or more embodiments, processor 123 may calculate a mathematical correlation between the transmission risk index and physical dimensions of the point of interest and/or whether that particular point of interest contributed to a previous outbreak of the disease. For example, in an embodiment, when the transmission risk index of a particular disease is very high, e.g., the transmission rate of a disease having a R₀ greater than 10, the business risk index may be between 90-95 (out of a scale of 100), if the point of interest has small physical dimensions, e.g., less than 1000 square feet, and/or low air flow rate, e.g., due to HVAC design, but high mobility data, e.g., visits by 2,000 people per day. Similarly, the business risk index may be between 40-60 if the transmission risk index of a particular disease is moderate, e.g., transmission rate of the disease having a R₀ between 2-3, and the point of interest has large physical dimensions, e.g., more than 10,000 square feet with low to moderate mobility, e.g., between 100 to 500 visits per day. Block 440 may be followed by Block 460 and/or by block 450.

Block 450 (Identify Secondary Business Risk Index) may refer to processor 123 identifying a business risk index for a secondary point of interest which is a business risk for the transmission of the disease at a second point of interest. The business risk index is determined using a network based approach based on at least the mobility data, transmission risk data, and the point of interest data to build a model of the business risk for the transmission of the disease for secondary points of interest that are related to the primary point of interest. The model may be used to determine the disease dynamics using a regression based machine learning and Artificial Intelligence (AI) based predictive analytics approach based on the mobility data and the point of interest data to quantify the risks pertaining to the disease (or health risk). For example, in an embodiment, business risk index for a secondary point of interest may be between 60-80 depending on the transmission risk index of a particular disease and if the secondary point of interest is a point of interest that is likely to be visited after visiting a primary point of interest, e.g., the primary point of interest being a children's clothing store and the secondary point of interest being a children's toy store or the primary point of interest being a doctor's office and the secondary point of interest being a pharmacy. Block 450 may be followed by Block 460.

In one or more embodiments, the business risk index may be determined based on the SEIR model and/or any additional transmission model(s) for the disease, mobility data, point of interest data, and Census block group data. For example, the mobility data may include data on hourly visits of a population of a Census block group to a point of interest, in which each Census block group has its own SEIR component, e.g., particular disease transmission among a certain group based on, for example, area, ethnicity, age, gender, etc. The type of infections from the disease may also be divided into the categories of infections for the Census block group or the point of interest. Using this data, a joint distribution of the Census block group mobility to certain points of interest, where fitting algorithms may be used, for example, an iterative proportional fitting algorithm. The business risk index may then be determined from test data that includes the point of interest data, e.g., point of interest area, layout, dimensions, mobility data, e.g., median dwelling time, and transmission risk data, e.g., time varying infectious population density. The business risk index determination may then be validated based on daily case counts from the disease data, e.g., local disease tracking. Thus, by determining the business risk index to get infection counts at each point of interest, the points of interests may be marked as highly contagious and in spreader categories. Accordingly, the points of interest that are marked as highly contagious may include strategies to mitigate the spread and transmission of the disease, for example, by reducing maximum occupancy of the point of interest, but not necessarily decreasing mobility to reduce the number of infections as it affects the time varying point of interest occupancy density. Thus, as discussed above, the business risk index may be used for reverse contact tracing to determine where disease transmission likely occurred and where disease transmission is likely to occur. In so doing, once a disease has spread to the community level (or some indication that the disease is likely to spread to the community), proactive mitigation strategies and responses may be implemented.

In another embodiment, it is appreciated that the Census block group may relate to a blocks of groups from non-U.S. countries and data associated therewith. Thus, the mobility data may be associated with the Census block group for visitors from foreign countries of origin. For example, the mobility data may include the number of visits, median medium dwell times, bucketed dwell times, e.g., time spans and number of visits, how often and in what hours the Census block group visits a particular point of interest in the same day, same week, etc. In so doing, the business risk index may be determined to rank points of interests and/or categories of businesses using the mobility data to identify the risks associated from each visitor from a Census block group to points of interest and calculate the transition of category from the S, E, I, and R categories. In another embodiment, the business risk index may be used to calculate the transition probability of individuals from a geographical location to a point of interest and then use linear regression to calculate the estimated cumulative cases at the point of interest.

In one or more embodiments, the predictive model(s) may have a plurality of analyser channels (customized for the disease and/or point of interest), each of which corresponds to an observable condition of the disease and/or point of interest. The channels may be weighted to customize or fine tune the predictive model(s), signifying whether any channels are of equal or greater/lesser importance than others in identifying the primary and/or secondary business risk index.

Predictive modelling may allow allocation of channel points in accordance with, or independent of, channel weighting based on the statistical sensitivity of specific factors in predicting, for example, the type of disease at the primary and/or secondary point of interest. For example, a predictive base score may be calculated as the summation of points attributed to the (weighted or unweighted) analyser channels. The analyser channels may be broken down further into analyser features that provide additional sensitivity in identifying points of interest(s) that may be at high/low risk of contributing to the transmission of the disease. For example, in an embodiment, if the primary point of interest is a movie theatre and the mobility data suggests that a likely secondary point of interest is a drive-thru burger restaurant, this secondary point of interest may be weighted less since there is less opportunities for contact and transmission of the disease, e.g., by infected driver to restaurant operator.

In one or more embodiments, points and/or weights may be assigned to each channel. It should be noted that not all of the channels or features need to be part of any given analysis. Moreover, other channels and/or features may be suitable in addition or in the alternative, depending on the study or analysis. In one or more embodiments, point modifiers may be applied to one or more of the channels and/or features to affect the influence of the same on the total predictive base score. Non-limiting examples include percentage weightings, inclusion/exclusion of certain channels/features to suit any particular analysis or subject point of interest, etc.

In one or more embodiments, the model may place a higher weight or point value for any or all of the channels. In other words, whether certain points of interests are at greater risk of transmitting the disease. For example, a grocery store may be a point of interest that has a greater risk of transmission than a gym. Accordingly, informed guidance may be given towards mitigation strategies.

Thus, Block 460 (Adjust Model) may refer to one or more channels being modified, deleted, or added to the predictive model(s) (starting from an initial model, e.g., in a recursive algorithm) and fed back to one of respective Blocks 440 or 450 for re-testing in an iterative process performed until Block 460 is answered “YES.” For example, a channel may be modified by adding points or point multipliers, or by changing or adding the weighting until the predictive score matches or exceeds a predetermined threshold, e.g., between a 90-95% confidence interval.

The predictive score may be adjusted based on several variables in order to obtain a business risk index that may be used to determine and/or adjust the mitigation strategy, for example. In one or more embodiments, a positive adjustment may be made based on the number of total active channels as well as having channels with greater than five active analyser features. A negative adjustment may be made for single active channels as well as for having fewer than five active features among all analyser channels.

Block 470 (Output the Model) may refer to processor 123 outputting the predictive model for, e.g., incorporation into a response manager or decision maker controlled computer or web-enabled interactive dashboard, as the model may be considered to be valid for implementation in determining whether the point of interest is a risk for spreading the disease. It is appreciated that the model can be used for the point of interest that was inputted into the training of the model and/or used for points of interest that have similar layouts, dimensions, foot traffic, occupant density, business category, e.g., dining restaurant, etc.

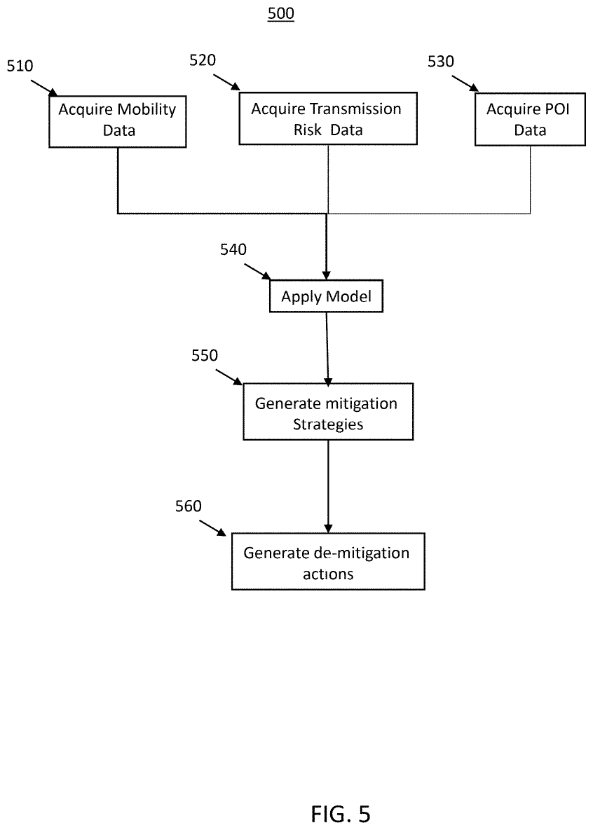

FIG. 5 shows an example processing flow 500 for a response engine to facilitate generating and/or adjusting mitigation strategies, according to at least one example embodiment described herein. In one or more embodiments, the model uses current and/or predicted disease data at the local level to generate and/or adjust mitigation strategies. The model may also use point of interest data (point of interest layout, dimensions, category, etc.) to generate the mitigation strategies. It is appreciated that the model may be used to determine the business risk index for the same or similar disease and/or the same or similar point of interest.

Processing flow 500 may include one or more operations, actions, or functions depicted by one or more blocks 510, 520, 530, 540, 550, and 560. Although illustrated as discrete blocks, various blocks may be divided into additional blocks, combined into fewer blocks, or eliminated, depending on the desired implementation. As a non-limiting example, the description of processing flow 500, corresponding to the depiction thereof in FIG. 5 and performed by processing system 120 in one or more embodiments described herein, pertains to generating and/or adjusting mitigation strategies under a certain condition (e.g., for a particular location, under a certain temperature, etc.). Processing may begin at blocks 510, 520, and 530.

Block 510 (Acquire Mobility Data) may refer to processing system 120 receiving a first set of mobility data from data sources 110A . . . N via communication ports 127. The data acquired in Block 510 may include, without limitation, mobility and foot traffic data for a given locality, state, region, or country based on, for example, cell phone data, sales data, satellite data, Wi-Fi signals, automobile data, proximity/pressure sensors, cameras, manual counters, etc. The data may be acquired via wireless or wired communications from data sources 110A . . . N or manual entry (for example, by an operator using a keyboard or tablet, smartphone, etc. utilizing appropriate application software). Block 510 may be followed by Block 540.

Block 520 (Acquire Point of Interest Data) may refer to processing system 120 receiving point of interest(s) data from data sources 110A . . . N via communication ports 127. The data acquired in Block 520 may include, without limitation, a layout and geospatial visualizations, dimensions and/or geodesic area, mechanical equipment installed at the point of interest, e.g., HVAC system and capacity, capacity, category type of the point of interest, e.g., restaurant, retail, parks, museums, bank, hotels, grocery stores, malls, houses of worship, etc. The data may be acquired via wireless or wired communications from data sources 110A . . . N or manual entry (for example, by an operator using a keyboard or tablet, smartphone, etc. utilizing appropriate application software). In some embodiments, and without limitation, the point of interest data may be obtained from public sources such as the government agency or online sources. Block 520 may be followed by Block 540.

Block 530 (Acquire Transmission Risk Data) may refer to processing system 120 receiving a set of transmission risk data from data sources 110A . . . N via communication ports 127. The data acquired in Block 530 may include transmission risk indices for the disease or identifiable vectors of transmission. The data may be acquired via wireless or wired communications from data sources 110A . . . N or manual entry (for example, by an operator using a keyboard or tablet, smartphone, etc. utilizing appropriate application software). In some embodiments, and without limitation, the transmission risk data may be obtained from public sources such as the government agency, the Centers for Disease Control, etc. Block 530 may be followed by Block 540.

Block 540 (Apply Model) may refer to processor 123 analysing the data acquired in Blocks 510, 520, and 530 in accordance with the model(s) validated according to procedure 400 and outputted at Block 470. For example, the data in each analyser channel may be converted to a channel score. The acquired data may be related to a disease that has the same or similar vectors of transmission and/or disease dynamics and/or a point of interest that has the same or similar layout, dimensions, etc. that are applied to the model(s). That is, the model may be used to determine yesterday where today's disease came from without contact tracing. Block 540 may be followed by Blocks 550 and/or 560.

Block 550 (Generate Mitigation Strategies) may refer to processor 123 generating plans for mitigating the transmission of the disease based on the channel scores determined in Block 540. For example, the channel scores may be summed to determine which mitigation strategies/responses should be taken. Based on the business risk index and the channel scores, mitigation strategies may be implemented for targeted responses to prevent or mitigate the spread of the disease. For example, if the business risk index exceeds a predetermined threshold, e.g., greater than 60 out of a scale of 100, the model may be used to automatically implement mitigation responses at that particular point of interest or inform decisions makers of what actions may need to be taken, e.g., mitigation strategies that are likely necessary to prevent and/or mitigate the transmission of the disease. Such mitigation strategies/responses may include decreasing hours of operation, decreasing occupant density, closing the point of interest, decreasing dwelling time at certain locations, e.g., moving people who were eating at one location to have after dinner dessert or coffee at another location at the point of interest, blocking off of certain areas at the point of interest, e.g., the bar at a restaurant, changes and improvements to the built environment, e.g., heating and cooling, air flow, and air circulation systems, etc. The targeted approach to mitigating the transmission of the disease allows maintaining a controlled opening of the economy instead of closing all the points of interests, e.g., during a pandemic. That is, points of interests that have a low business risk index, e.g., less than 50 out of a scale of 100, may remain open without restrictions, while the points of interest that have a high business risk index, e.g., greater than 60, are controlled with mitigation strategies, and if having a business risk index of greater than a predetermined maximum, e.g., 95, closing/locking down the point of interest.

Block 560 (Generate De-Mitigating Actions) may refer to processor 123 generating plans for de-mitigating based on the channel scores determined in Blocks 540. For example, the channel scores may be summed to create de-mitigating actions. The de-mitigating actions include developing plan for structured strategies/responses for re-opening/removing restrictions for any or all impacted points of interest(s) that have reduced business risk indexes, e.g., pandemic is near an end. For example, if the transmission risk index decreases, for example, due to vaccinations and/or mobility data decreases, e.g., seasonal changes, the business risk index for a point of interest may decrease. When the business risk index reaches certain predetermined minimum values, e.g., decreases from 90 to 60, the response engine may direct actions to de-mitigate the plans, e.g., increase occupant density restrictions, remove store hour limits, etc. It is appreciated, however, that the BRI may be used with other indexes for determining overall risks to public health. For example, while the BRI for a disease may be used to determine the mitigation actions for a community in general, the severity of the disease may also be used to determine when actions may be taken and when the actions should be taken. For example, if the mortality risk of a disease is high, e.g., for a certain age group or demographic, the Mortality Risk Index (MRI) may be used to weight the BRI and/or be used in conjunction with the BRI. In an embodiment, if the MRI which identifies the risk of morality or criticality of the disease is high, mitigating actions may be taken at lower BRI values, e.g., taking actions at lower BRI values for high MRI disease relative to areas or diseases with a low MRI value.

It is appreciated that in an embodiment, the business risk index may also be used in a variety of ways. For example, the business risk index may be used to determine certain factors of a point of interest that may contribute to the likelihood of a transmission outbreak, e.g., type of business and/or certain geodesic area. The points of interest that are found to have the determined factors may be proactively altered, e.g., moving a host check-in station to have multiple bays or areas at a restaurant to avoid dwelling time or altering movement of people within a point of interest.

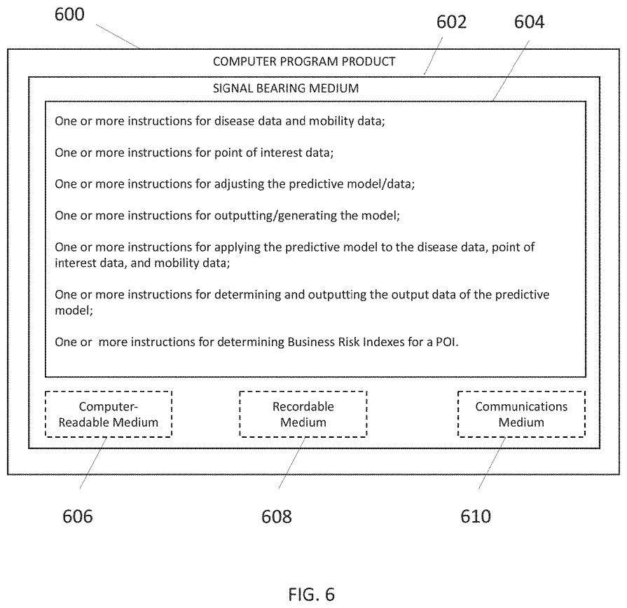

FIG. 6 illustrates at least one computer program product that may be utilized to provide the response engine, according to at least one example embodiment described herein. Program product 600 may include a signal bearing medium 602. Signal bearing medium 602 may include one or more instructions 604 that, when executed by, for example, a processor, may provide the functionality described above with respect to FIGS. 4-5. By way of example, but not limitation, instructions 604 may include: one or more instructions for disease data and mobility data, one or more instructions for point of interest data, one or more instructions for geocoding the data, one or more instructions for adjusting the predictive model/data, one or more instructions for outputting/generating the model, one or more instructions for applying the predictive model to the disease data, point of interest data, and mobility data, one or more instructions for determining and outputting the output data of the predictive model, one or more instructions for determining business risk index for a point of interest, etc. Thus, for example, referring to FIGS. 4-5, processor 123 may undertake one or more of the blocks shown in FIGS. 4-5 in response to instructions 604.

In some implementations, signal bearing medium 602 may encompass a computer-readable medium 606, such as, but not limited to, a hard disk drive, a CD, a DVD, a flash drive, memory, etc. In some implementations, signal bearing medium 602 may encompass a recordable medium 608, such as, but not limited to, memory, read/write (R/W) CDs, R/W DVDs, etc. In some implementations, signal bearing medium 602 may encompass a communications medium 610, such as, but not limited to, a digital and/or an analog communication medium (e.g., a fiber optic cable, a waveguide, a wired communications link, a wireless communication link, etc.). Thus, for example, computer program product 600 may be conveyed to one or more modules of processor 123 by an RF signal bearing medium, where the signal bearing medium is conveyed by a wireless communications medium (e.g., a wireless communications medium conforming with the IEEE 802.11 standard).

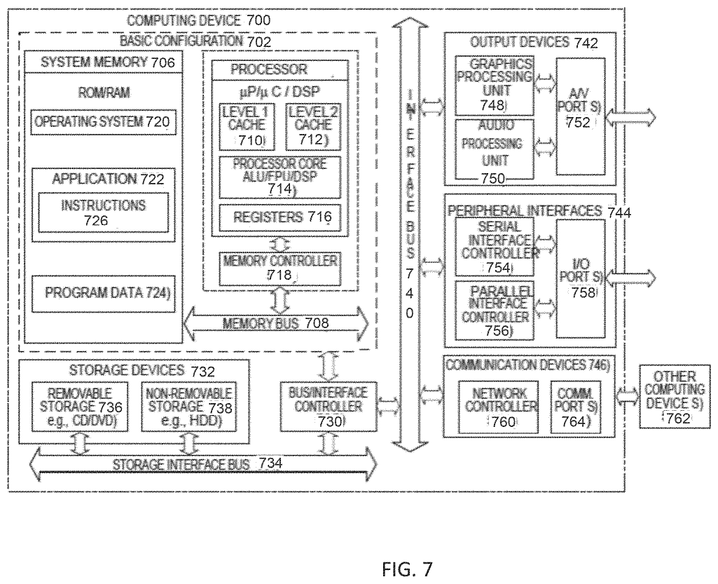

FIG. 7 shows a block diagram illustrating an example computing device 700 by which various example solutions described herein may be implemented, according to at least one example embodiment described herein. In a very basic configuration 702, computing device 700 typically includes one or more processors 704 and a system memory 706. A memory bus 708 may be used for communicating between processor 704 and system memory 706.

Depending on the desired configuration, processor 704 may be of any type including but not limited to a microprocessor (μP), a microcontroller (μC), a digital signal processor (DSP), or any combination thereof. Processor 704 may include one or more levels of caching, such as a level one cache 710 and a level two cache 712, a processor core 714, and registers 716. An example processor core 714 may include an arithmetic logic unit (ALU), a floating point unit (FPU), a digital signal processing core (DSP Core), or any combination thereof. An example memory controller 718 may also be used with processor 704, or in some implementations memory controller 718 may be an internal part of processor 704.

Depending on the desired configuration, system memory 706 may be of any type including but not limited to volatile memory (such as RAM), non-volatile memory (such as ROM, flash memory, etc.) or any combination thereof. System memory 706 may include an operating system 720, one or more applications 722, and program data 724. Application 722 may include instructions 726 to carry out predicting a spread of a disease, and predicting and responding to points of interest at risk of spreading the disease that are arranged to perform functions as described herein including those described with respect to process 400, 500 of FIGS. 4-5. Program data 724 may include data (e.g., population, mobility, point of interest data, etc.) from data resources 110A . . . N that may be useful for the response engine as is described herein. In some embodiments, application 722 may be arranged to operate with program data 724 on operating system 720 such that implementations of the response engine in, e.g., government entity or point of interest manager, may be provided as described herein. This described basic configuration 702 is illustrated in FIG. 7 by those components within the inner dashed line.

Computing device 700 may have additional features or functionality, and additional interfaces to facilitate communications between basic configuration 702 and any required devices and interfaces. For example, a bus/interface controller 730 may be used to facilitate communications between basic configuration 702 and one or more data storage devices 732 via a storage interface bus 734. Data storage devices 732 may be removable storage devices 736, non-removable storage devices 738, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and non-volatile, removable and non-removable media implemented in any method or technology for storage of information, such as computer readable instructions, data structures, program modules, or other data.

System memory 706, removable storage devices 736 and non-removable storage devices 738 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 700. Any such computer storage media may be part of computing device 700.

Computing device 700 may also include an interface bus 740 for facilitating communication from various interface devices (e.g., output devices 742, peripheral interfaces 744, and communication devices 746) to basic configuration 702 via bus/interface controller 730. Example output devices 742 include a graphics processing unit 748 and an audio processing unit 750, which may be configured to communicate to various external devices such as a display or speakers via one or more A/V ports 752. Example peripheral interfaces 744 include a serial interface controller 754 or a parallel interface controller 756, which may be configured to communicate with external devices such as input devices (e.g., keyboard, mouse, pen, voice input device, touch input device, etc.) or other peripheral devices (e.g., printer, scanner, etc.) via one or more I/O ports 758. An example communication device 746 includes a network controller 760, which may be arranged to facilitate communications with one or more other computing devices 762 over a network communication link via one or more communication ports 764.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RE), microwave, infrared (IR) and other wireless media. The term computer readable media as used herein may include both storage media and communication media.

Computing device 700 may be implemented as a portion of a small-form factor portable (or mobile) electronic device such as a cell phone, a tablet, a personal data assistant (PDA), a personal media player device, a wireless web-watch device, a personal headset device, an application specific device, or a hybrid device that include any of the above functions. Computing device 700 may also be implemented as a personal computer including both laptop computer and non-laptop computer configurations.

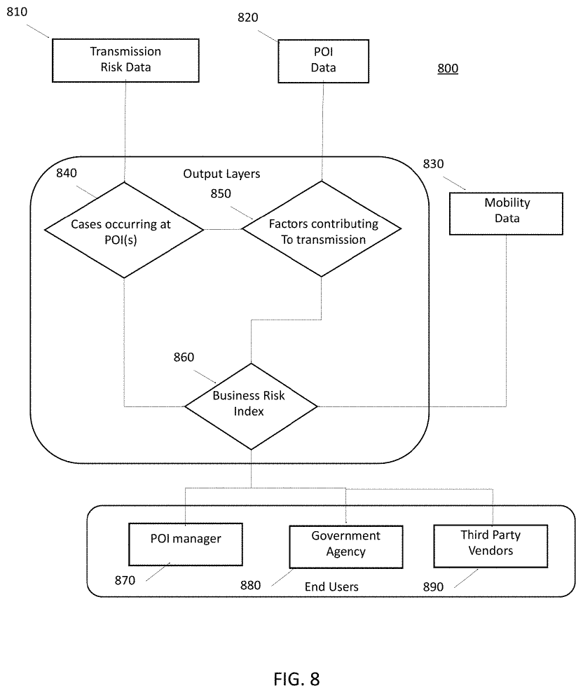

FIG. 8 illustrate a work flow 800 of a response engine, according to at least one example embodiment described herein.

As shown in FIG. 8, Blocks 810, 820, 830 represent inputs to the response engine; Blocks 840, 850, 860 represent outputs of the response engine, and Blocks 870, 880, and 890 represent users of the output of the response engine. It will be appreciated that the input Blocks 810, 820, 830, can be e.g., data sources 110A . . . N of FIG. 1; and the users Blocks 870, 880, and 890 can be e.g., the decisions makers 140 of FIG. 1. Input data may further include e.g., environmental data such as air quality data, temperature data, humidity data, etc.

In at least one example embodiment, Block 810 represents transmission risk data, including disease (e.g., COVID-19) case statistics and dynamics. Block 820 represents point of interest data including the layout and geospatial visualizations, dimensions and/or geodesic area, category type of the point of interest, e.g., restaurant, retail, parks, museums, bank, hotels, grocery stores, malls, houses of worship, etc. Block 830 represents population mobility data, e.g., mobility and foot traffic data for a given locality, state, region, or country based on, for example, cell phone data, sales data, satellite data, Wi-Fi signals, automobile data, proximity/pressure sensors, cameras, manual counters, etc. of the subjects, e.g., people in the community.

In at least one example embodiment, Block 840 represents cases of the disease by point of interest in a particular area, Block 850 represent factors of the point of interest that are determined to contribute to transmission of the disease, and Block 860 represents Business Risk Index of the disease. In at least one example embodiment, the accessibility of the output Blocks 870, 880, 890 of the response engine include online access of the response engine, web browser, and mobile devices. The output may be open to public and/or licensed to specific users; output data sets may be available through the application programming interfaces, the web feature services having a log-in to a dashboard, the web map services, and/or direct download. The response engine provides friendly user interactive interface to visualize and query data, may be hosted on web services, and may be replicated in a local environment for additional privacy and security. For example, in an embodiment, the business risk index may be used to rank points of interests based on the risk of transmission and to determine which points of interest are contributing to a major portion of the disease transmission. The business risk index mays also be used to determine the number of potential transmissions happening at that business, e.g., based on how densely a point of interest is occupied by its visitors. The response engine may also provide statistics for total visits (daily/monthly/yearly), average dwelling time, transmissions at each POI for different categories of POI at county level as well as for category of businesses for the top-ranked BRIs.

While the response engine is accessible by decision makers, it is also appreciated that third party vendors may also have access to the BRI to be able to target certain points of interest that may need to update/upgrade certain aspects to reduce the chance of spread of the disease. For example, an HVAC company may find a particular point of interest has a high BRI based on the past spread of a disease and HVAC system installed and layout/dimensions of the point of interest. The HVAC company may then target the point of interest to update/upgrade the HVAC system, e.g., increase capacity, increase flow rates, etc.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

For example, it is appreciated that the business risk index may be used for determining future zoning for a particular geographical location. In an embodiment, the business risk index used to determine and track the potential spread of a disease may be used to consider zoning of particular areas and the effect of zoning, e.g., residential, commercial, etc., on the potential spread or transmission of the disease to individual buildings, communities, and to particular Census block groups.

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A method of determining and tracking spread of at least one disease at a plurality of points of interest, comprising:
geocoding movement data and health information of a plurality of subjects acquired from a plurality of sources;
merging the geocoded movement data and the geocoded health information;
obtaining point of interest information of the plurality of points of interest from the plurality of sources;
developing, for each of the plurality of points of interest, a business risk index of the risk of transmission of the at least one disease at the plurality of points of interest based on the merged geocoded movement data, the geocoded health information, and the point of interest information;
outputting the business risk index for the plurality of points of interest; and
developing and adjusting strategies to mitigate the disease based on the business risk index.

2. The method according to claim 1, wherein the geocoded health information includes a transmission risk of the at least one disease in a community that includes the plurality of subjects.

3. The method according to claim 1, wherein the point of interest information includes physical dimensions of the point of interest.

4. The method according to claim 1, wherein the strategies include controlling occupant density within the at least one point of interest or locking down the at least one point of interest.

5. The method according to claim 1, wherein the at least one disease is an infectious disease or a non-infectious disease.

6. The method according to claim 1, further comprising identifying points of interest that have the highest business risk index for the plurality of points of interest.

7. The method according to claim 1, further comprising determining, based on the geocoded movement data, an average dwelling time at at least one of the plurality of points of interest or a business category of the plurality of points of interest that had the most visitations.

8. The method according to claim 1, wherein the developing and adjusting strategies to mitigate the disease based on the business risk index automatically triggers a health risk review of at least one of the plurality of points of interest.

9. The method according to claim 1, wherein the developing and adjusting strategies to mitigate the disease based on the business risk index includes automatically limiting an occupancy density or lock-down of the at least one point of interest when the business risk index of the at least one point of interest exceeds a predetermined value.

10. A system comprising:
a memory to store structured information and unstructured information; and
a processor configured to:
geocode movement data of a plurality of subjects acquired from a plurality of sources;
geocode health information of the plurality of subjects from the plurality of sources;
merge the geocoded movement data and the geocoded health information;
obtain point of interest information of the plurality of points of interest from the plurality of sources,
develop, for each of the plurality of points of interest, a business risk index of the risk of transmission of the at least one disease at the plurality of points of interest based on the merged geocoded movement data, the geocoded health information, and the point of interest information;
output the business risk index for the plurality of points of interest; and
request a user to develop and adjust strategies to mitigate the disease based on the business risk index.

11. The system according to claim 10, wherein the strategies include controlling occupant density within the at least one point of interest or locking down the at least one point of interest.

12. The system according to claim 10, wherein the request is a request to automatically trigger a health risk review of at least one of the plurality of points of interest.

13. The system according to claim 10, wherein the request is a request to automatically limit an occupancy density or lock-down the at least one point of interest when the business risk index of the at least one point of interest exceeds a predetermined value.

14. A non-transitory computer-readable medium having computer-readable instructions that, when executed by a computing device, cause the computing device to perform operations comprising:
geocoding movement data of a plurality of subjects acquired from a plurality of sources;
geocoding health information of the plurality of subjects from the plurality of sources;
merging the geocoded movement data and the geocoded health information;
obtaining point of interest information of the plurality of points of interest from the plurality of sources,
developing, for each of the plurality of points of interest, a business risk index of the risk of transmission of the at least one disease at the plurality of points of interest based on the merged geocoded movement data, the geocoded health information, and the point of interest information;
outputting the business risk index for the plurality of points of interest; and
developing and adjusting strategies to mitigate the disease based on the business risk index.

15. The non-transitory computer-readable medium according to claim 14, wherein the strategies include controlling occupant density within the at least one point of interest or locking down the at least one point of interest.

16. The non-transitory computer-readable medium according to claim 14, wherein the developing and adjusting strategies to mitigate the disease based on the business risk index automatically triggers a health risk review of at least one of the plurality of points of interest.

17. The non-transitory computer-readable medium according to claim 14, wherein the developing and adjusting strategies to mitigate the disease based on the business risk index includes automatically limiting an occupancy density or lock-down the at least one point of interest when the business risk index of the at least one point of interest exceeds a predetermined value.
